(19) United States
(12) Patent Application Publication
(10) Pub. No.: US 2024/0284998 A1
(43) Pub. Date: Aug. 29, 2024

(54) Discrete Ostomy-accessible Clothing

(71) Applicant: Willa Dorrough, McDonough, GA (US)

(72) Inventor: Willa Dorrough, McDonough, GA (US)

(73) Assignee: VerityWear Pajamas, LLC, McDonough, GA (US)

(21) Appl. No.: 18/113,429

(22) Filed: Feb. 23, 2023

(51) Int. Cl. A41D 13/00; A41D 13/12; A61F 5/445

(52) U.S. Cl. CPC A41D 13/0012; A41D 13/1254; A61F 5/445

(57) ABSTRACT

The present disclosure provides generally for ostomy-accessible clothing, particularly sleepwear. According to the present disclosure, the ostomy-accessible clothing may comprise one or more openings. The openings may be configured to adapt to a stoma pouch, wherein the stoma pouch is fitted with or without an external device, such as a drainage bag. The openings may be located on either side of the ostomy-accessible clothing to provide access to a stoma bag. The ostomy-accessible clothing may comprise at least one fastening mechanism. The ostomy-accessible clothing may comprise one or more storage compartments. The stoma pouch may interface with an external device by means of a connecting apparatus. The ostomy-accessible clothing may comprise an elastic portion. When the ostomy-accessible clothing comprises an elastic portion, the elastic portion may comprise an opening. When the elastic portion comprises an opening, the elastic portion's tension may maintain a closed state.

### BACKGROUND

In the United States alone, there are over 100,000 ostomies performed each year, with thousands more being performed annually around the world. An ostomy is a surgical procedure that is performed to reroute the human body's natural bodily waste pathway. These procedures are performed for a variety of reasons. Most commonly, ostomies are performed when parts of the bladder or bowels need time to heal or need to be removed. The most common ostomies are urostomies, typically performed on the ileum or cecum; colostomies, which are typically performed on the colon or rectum; and ileostomies, which are performed on the ileum.

Ostomy procedures can be either permanent or temporary, and result in the creation of a surgical opening called a stoma. During surgery, an opening is created along the bodily waste path, on the small or large intestine. The surgeon then pulls and sews the opening in the intestine to the patient's abdomen, resulting in the formation of the stoma. After the procedure is performed and the stoma is created, the patient attaches a pouch to the stoma to receive waste that would normally pass through the body. For example, a urostomy, is an ostomy that is performed on the small intestine to reroute the path of urine, and instead of passing through the bladder, urine will pass through the stoma and into the pouch.

Stomas are typically located on a patient's lower abdomen on the left or right side. The stoma pouch therefore extends over much of the patient's lower abdomen and pelvis. Because of the size and location of the stoma pouch, the patient's ability to wear pants is affected, especially at night. During the night, ostomy patients typically attach a drainage bag to their pouch, so that they do not have to worry about changing their pouch in the middle of the night. The stoma pouch already takes up quite a bit of space on the patient's abdomen and the drainage bag adds further complications to the patient's ability to comfortably wear pants or other types of sleepwear. When a drainage bag is attached to a stoma pouch, the patient may have to pull their pants down significantly to accommodate the drainage bag. Drainage bag use is not only reserved for nighttime use, so when a patient utilizes a drainage bag during the day, not only is their ability to comfortably wear clothes affected but it may also affect them emotionally if they have no choice but to wear their clothes in a compromising fashion.

Currently there exists very few alternatives for ostomy patients to comfortably wear their normal fashion wear without experiencing the inconveniences encountered when wearing a stoma pouch and drainage bag. Ostomy patients do not yet have the luxury of wearing clothing that provides easy access to the stoma pouch for purposes of changing it or for medical professionals to perform examinations.

Comfort and dignity are two of the most important aspects of recovery, and someone's medical condition should not get in the way of their ability to feel comfortable and confident. Especially in the case where a patient receives a permanent stoma, there is no reason that they should have to go the rest of their lives sacrificing comfort. Not only do ostomy patient's find themselves sacrificing comfort at night, but they encounter this problem at the hospital and doctor's office as well.

When an ostomy patient goes in for a routine check-up or emergency visit, because of the location of the stoma and stoma pouch, the doctor may need them to remove their pants to view the stoma and stoma pouch. This is not only inconvenient, but many people would prefer not to disrobe in front of doctors and maintain their modesty. Ostomies are performed on patients of all different sizes, ages, and genders so it is important that they have the benefit of maintaining their modesty to the level of which they are most comfortable with.

### SUMMARY OF THE DISCLOSURE

What is needed is a comfortable and convenient way for ostomy patients to wear clothing in a manner that makes the stoma pouch accessible, while also not interfering with the wearability of the patient's clothing. An adaptable article of clothing that provides access to an ostomy patient's stoma pouch would eliminate problems caused when wearing clothing with a stoma pouch.

By comprising an opening, the adaptable clothing provides a way for an ostomy patient to wear a drainage bag without the patient being forced to wear their pants, for example, in an uncomfortable or immodest fashion, particularly at night. The opening also addresses the issues encountered during doctor visits, by presenting easy access to the stoma pouch, the adaptable clothing with the opening may eliminate the need for undressing for a doctor examination.

The present disclosure provides generally for ostomy-accessible clothing. According to the present disclosure, the device may comprise one or more openings on an article of clothing. The ostomy-accessible clothing may comprise various materials and may conform to various shapes and sizes. The openings may be configured to adapt to a stoma pouch, wherein the stoma pouch is fitted with or without a drainage bag. The openings may be located on either side of the ostomy-accessible clothing to provide access to an ostomy patients stoma bag.

In some embodiments, the ostomy-accessible clothing may comprise at least one fastening mechanism. In some implementations, the ostomy-accessible clothing may comprise one or more storage compartments. In some aspects, the stoma pouch may interface with an external device by means of a connecting apparatus.

In some embodiments, the ostomy-accessible clothing may comprise an elastic portion. In some implementations, when the ostomy-accessible clothing comprises an elastic portion, the elastic portion may comprise an opening. In some aspects, when the elastic portion comprises an opening, the elastic portion's tension may maintain a closed state.

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1A illustrates a closed state of an exemplary ostomy-accessible clothing, according to some embodiments of the present disclosure.

FIG. 1B illustrates an open state of an exemplary ostomy-accessible clothing, according to some embodiments of the present disclosure.

FIG. 1C illustrates a closed state of an exemplary ostomy-accessible clothing, according to some embodiments of the present disclosure.

FIG. 1D illustrates an open state of an exemplary ostomy-accessible clothing, according to some embodiments of the present disclosure.

FIG. 2A illustrates a closed state of an exemplary ostomy-accessible clothing comprising an elastic portion, according to some embodiments of the present disclosure.

FIG. 2B illustrates an open state of an exemplary ostomy-accessible clothing comprising an elastic portion, according to some embodiments of the present disclosure.

FIG. 3 illustrates a closed state of an exemplary ostomy-accessible clothing comprising at least one storage compartment, according to some embodiments of the present disclosure.

FIG. 4A illustrates a closed state of an exemplary ostomy-accessible clothing comprising a fastening mechanism, according to some embodiments of the present disclosure.

FIG. 4B illustrates an open state of an exemplary ostomy-accessible clothing comprising a fastening mechanism, according to some embodiments of the present disclosure.

FIG. 5A illustrates an exemplary ostomy-accessible clothing, according to some embodiments of the present disclosure.

FIG. 5B illustrates an exemplary ostomy-accessible clothing, according to some embodiments of the present disclosure.

FIG. 5C illustrates an exemplary ostomy-accessible clothing, according to some embodiments of the present disclosure.

FIG. 5D illustrates an exemplary ostomy-accessible clothing, according to some embodiments of the present disclosure.

FIG. 6 illustrates an exemplary ostomy-accessible clothing providing an interface between a stoma pouch and an external device, according to some embodiments of the present disclosure.

### DETAILED DESCRIPTION

In the following sections, detailed descriptions of examples and methods of the disclosure will be given. The description of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

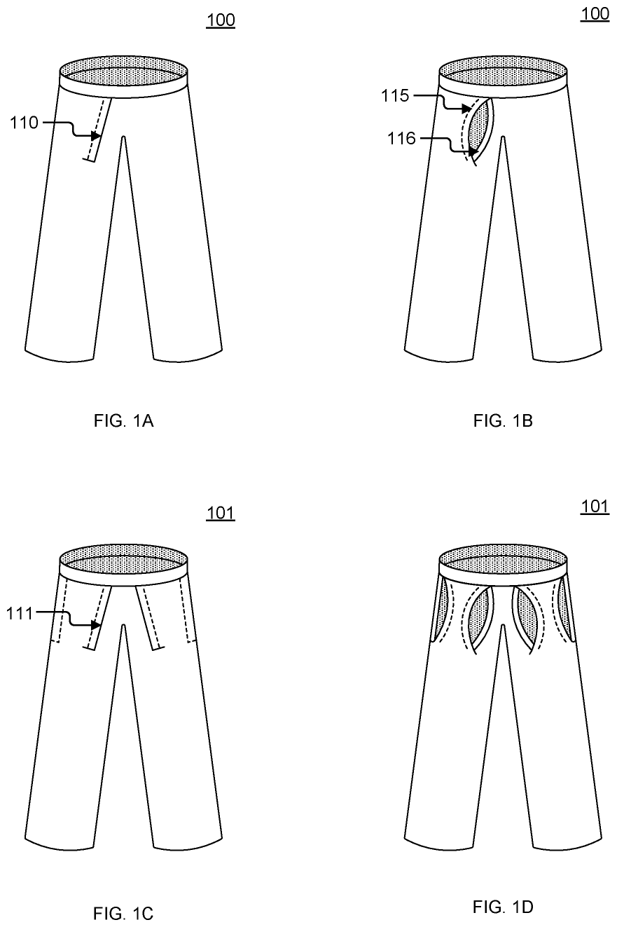

Referring now to FIGS. 1A-D, a closed state of an exemplary ostomy-accessible clothing 100, 101 is illustrated. In some embodiments, the ostomy-accessible clothing 100 may comprise at least one opening 110, 111.

In some implementations, the opening 110 may comprise a predefined slit in the ostomy-accessible clothing 100 that allows the maintenance and replacement of a stoma pouch. Because the stoma pouch typically extends from the lower abdominal to the pelvic region, accessing the stoma pouch in regular clothing is difficult and often requires the adjustment of clothing. Due to the physical location of the stoma pouch, adjustment of regular clothing to reach the stoma pouch may result in indecency or lack of dignity, even in private settings.

As a non-limiting example, wherein the wearable clothing comprises sleepwear, as a user sleeps, they may attach a drainage bag to their stoma. The ostomy-accessible clothing 100 may allow a user to sleep comfortably and fully covered without risk of detaching the bag or exposing their skin. As another illustrative example, if a person wearing the ostomy-accessible clothing 100 goes for a medical examination, such as at a hospital, the doctor will simply need to examine the stoma and stoma pouch through the opening 110 as opposed to completely disrobing for the examination.

In some aspects, the opening 110 may be positioned adjacent to the location of the stoma pouch. In some embodiments, the width of the opening 110 may comprise a predefined width at least the size of a stoma pouch. In some implementations, the location and size of the opening 110 may allow the user to access the stoma pouch without clothing adjustment. This may preserve the user's modesty in addition to improving the ease of access to the stoma pouch.

In some aspects, the opening 110 may comprise one or more layer of fabric. In some embodiments, the one or more layers of fabric may allow the opening 110 to remain discreet and relatively unnoticeable. In some implementations, the one or more layers of fabric may form an overlapping layer. In some aspects, the overlapping layer may comprise a sufficient length that the opening 110 remains in a closed state when the opening 110 is not accessed.

In some aspects, an ostomy-accessible clothing may comprise wearable clothing configured to at least partially cover a first stoma of a user; at least a first opening on the wearable clothing, wherein, when the ostomy-accessible clothing is worn, at least the first opening is located proximate to the first stoma, and wherein at least the first opening further comprises at least two layers of fabric 115, 116, wherein at least the two layers of fabric 115, 116 are configured to overlap when in a resting position and configured to provide at least the first opening 110 when at least the two layers are separated.

In some embodiments, the ostomy-accessible clothing 101 may comprise two or more openings 111. In some implementations, the two or more openings 111 may improve ease of access to the stoma pouch. For example, the stoma pouch may need to switch out for a second stoma pouch, which process may require the use of both hands. The utilization of two or more openings 111 may enable the user to ease manipulate and switch out the stoma bags, utilizing the two or more openings 111 to use both hands for the process.

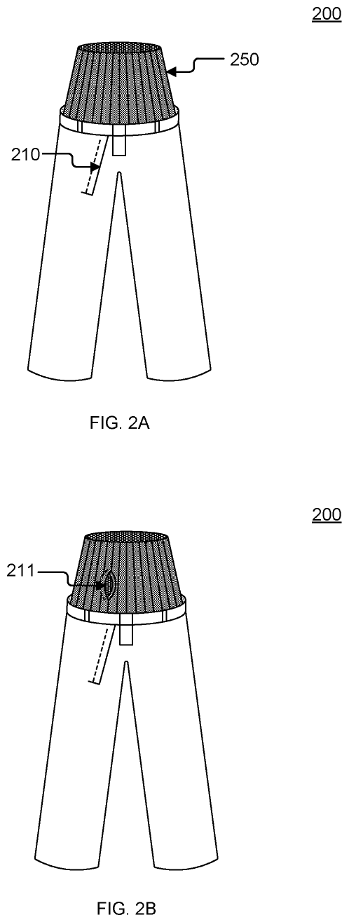

Referring now to FIGS. 2A-B, a closed state of an exemplary ostomy-accessible clothing comprising an elastic portion is illustrated. In some embodiments, the ostomy-accessible clothing 200 may comprise one or more openings 210, 211. In some implementations, the ostomy-accessible clothing 200 may comprise an elastic portion 250.

In some aspects, the elastic portion 250 may assist with maintaining alignment of the stoma pouch. In some embodiments, the elastic portion 250 may compress the stoma pouch against the body of the user sufficiently to make the stoma pouch indiscernible under regular clothing. Commonly, wearing looser fitting or specialized clothing has been a necessity for people with stoma pouches because the bag is clearly noticeable above the waistband with normal clothing. In some aspects, the ostomy-accessible clothing may be worn at least partially under other clothing. As an example, the elastic portion 250 may extend from the waistline upwards and cover at least a portion of the navel, which may also be covered by a shirt.

In some implementations, the elastic portion 250 may provide a means of securing a stoma pouch without restricting its operation. For example, regular legwear with a predefined width around the waist may be restrictive and difficult to wear. This may be difficult for a stoma pouch due to the stoma pouch extending across the same region as the waist of the legwear. In contrast, the elastic portion 250 may provide enough stability to properly secure a stoma pouch without the risk of restricting flow from the stoma pouch through constriction. The elastic portion 250 may improve the ease of access to the stoma pouch and remove the need to wear the waistband of legwear exceedingly low, so as to be potentially revealing.

In some aspects, the ostomy-accessible clothing 200 may comprise two or more openings 210, 211. In some embodiments, the openings 210, 211 may improve accessibility and comfort in adjusting the stoma pouch. For example, after the stoma pouch is secured against the body by the elastic portion 250, it may be easier to access the stoma pouch without disrupting the positioning of the stoma pouch by accessing the stoma pouch through a second opening 211. To further illustrate the example, being seated and standing in various positions may also affect ease of access, wherein the secondary opening may provide access in alternative positions.

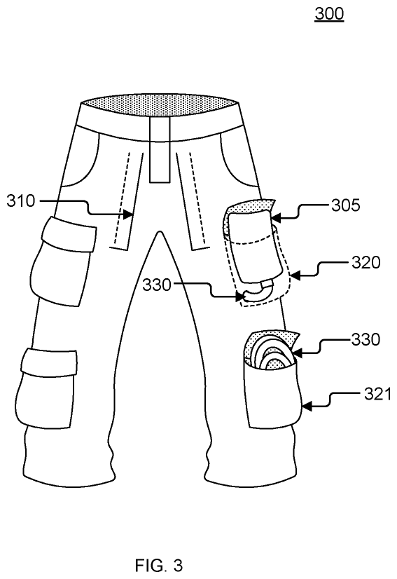

Referring now to FIG. 3, a closed state of an exemplary ostomy-accessible clothing 300 comprising at least one storage compartment 320 is illustrated. In some embodiments, the ostomy-accessible clothing 300 may comprise at least one opening 310. In some implementations, the ostomy-accessible clothing 300 may comprise one or more storage compartments 320, 321.

In some aspects, the storage compartments 320 may comprise an interior opening that allows items stored within the storage compartment to interface with the stoma pouch. For example, a supplemental pouch may be stored with a pocket on a pair of pants. The supplemental pouch may comprise tubing that connects to the stoma pouch. To further illustrate the example, ostomy patients typically utilize drainage bags which oftentimes comprise longer tubes. By utilizing the storage compartment 320, the drainage bag may pass through the pants to connect to the stoma pouch and allow the drainage bag to become portable by being stored in the patient's pants. The storage compartment 320 may comprise an opening on the interior side of the pocket that allows the tubing to connect from the pocket to the stoma pouch.

The interior access to the stoma pouch may prevent connecting tubes from visibility. In some embodiments, the interior access for connecting tubes may prevent snagging that may occur from loose, exterior tubes. For example, stoma drainage bag tubes can be rather lengthy, and by confining the tubes to the ostomy-accessible clothing 300, the patient will not have to worry about the tubes catching surfaces such as doorknobs, railings and other protrusions and obstacles.

In some implementations, the storage compartments 321 may allow supplemental materials to be stored in proximity to the stoma pouch. For example, an extra stoma pouch and other maintenance materials may be stored within a pocket on a pair of pants that allows the user to adjust and replace the stoma pouch as needed, regardless of location. In some aspects, the ostomy-accessible clothing may comprise two or more storage compartments 320, 321 to interface with the stoma pouch, store supplemental materials, or both, as a list of non-limiting examples.

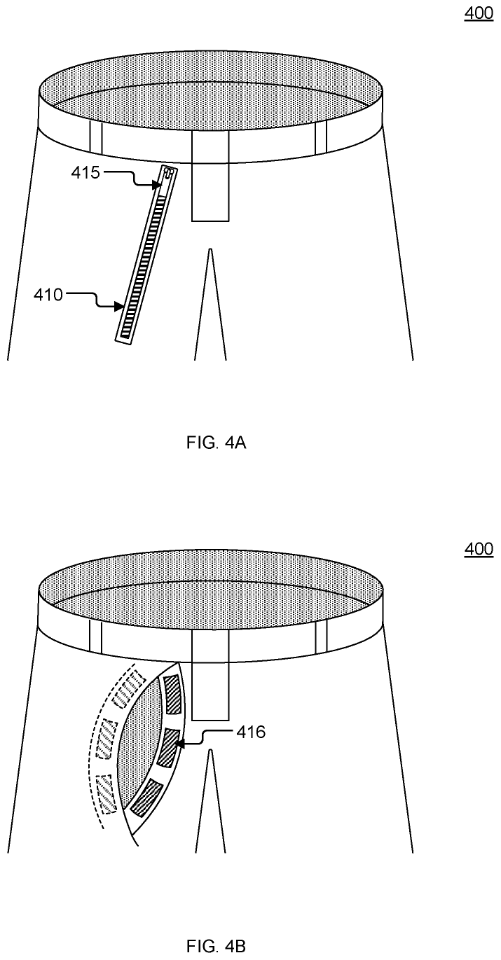

Referring now to FIGS. 4A-B, a closed state of an exemplary ostomy-accessible clothing comprising a fastening mechanism is illustrated. In some embodiments, the ostomy-accessible clothing 400 may comprise at least one opening 410. In some implementations, the opening 410 may comprise a fastening mechanism 415, 416.

By way of example and not limitation, the fastening mechanism 415, 416 may comprise at least one of: a hook, a clasp, a clip, a buckle, a zipper, a magnet, a hook and loop fastener, and a snap, or any similar fastening mechanism 415, 416, as well as any combination thereof. In some embodiments, the fastening mechanism 415, 416 may comprise an adjustment means that allows the size of the opening 410 to be altered.

In some implementations, the fastening mechanism 415, 416 may accommodate limited ranges of motion when accessing the opening 410. For example, a magnet fastener may be easier for a user who have an essential tremor than a buckle or a zipper. In some embodiments, the orientation, size, and position, as a non-limiting list of examples, of the opening 410 may similarly be adjusted to accommodate users with limited ranges of motion.

In some aspects, the opening 410 may comprise one or more layer of fabric. In some embodiments, the one or more layers of fabric may allow the opening 410 to remain discreet and relatively unnoticeable. In some implementations, the one or more layers of fabric may form an overlapping layer.

In some aspects, the overlapping layer may comprise a sufficient length that the opening 110 remains in a discrete closed state when the opening 410 is not accessed. In some implementations, the attaching surfaces of the fastening mechanism 415, 416 may be located on the interior adjacent surfaces of the opening 410 to maintain a discrete appearance.

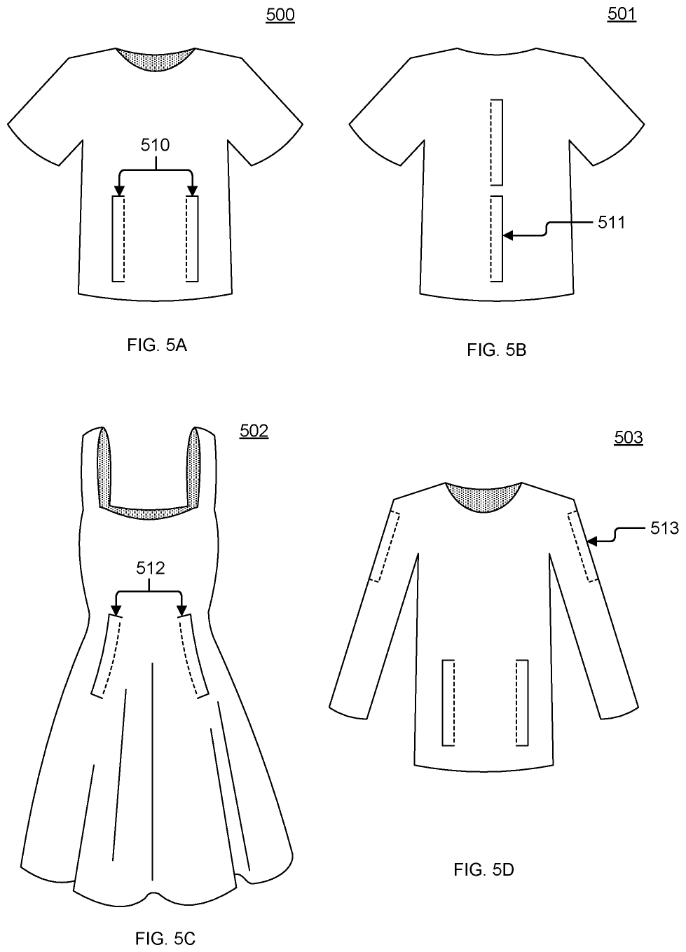

Referring now to FIG. 5A-D, an exemplary ostomy-accessible clothing 500, 501, 502, 503 is illustrated. In some embodiments, the ostomy-accessible clothing 500 may comprise one or more openings 510, 511, 512, 513.

In some aspects, an ostomy-accessible clothing may comprise a wearable clothing configured to at least partially cover one or more a first stoma, a second stoma, and a medical access point of a user. In some implementations, the ostomy-accessible clothing may comprise a plurality of openings 510, 511, 512, 513 on the wearable clothing, wherein, when the ostomy-accessible clothing is worn, each of the plurality of openings is located proximate to one of stomas or the medical access point.

In some implementations, the opening 510, 511, 512, 513 may comprise a predefined slit in the ostomy-accessible clothing 500, 501, 502, 503 that allows the maintenance and replacement of a stoma pouch. In some embodiments, the stoma pouch may comprise a pouch or implanted semi-permanent or permanent medical device that may require regular access and maintenance. In some implementations, accessing the stoma pouch in regular clothing is difficult and often requires the adjustment of clothing. Due to the physical location of the stoma pouch, adjustment of the clothing to reach the stoma pouch may result in indecency, especially in public settings. As an illustrative example, if a person wearing the ostomy-accessible clothing 500, 501, 502, 503 goes for a medical examination, the doctor may be able to examine the stoma and stoma pouch through the opening 510, 511, 512, 513 rather than requiring the person to disrobe.

In some aspects, the opening 510, 511, 512, 513 may be positioned adjacent to the location of the stoma pouch. In some embodiments, the width of the opening 510, 511, 512, 513 may comprise a predefined width at least the size of a stoma pouch. In some implementations, the location and size of the opening 510, 511, 512, 513 may allow the user to access the stoma pouch without clothing adjustment. This may preserve the user's modesty in addition to improving the ease of access to the stoma pouch.

In some aspects, the opening 510, 511, 512, 513 may comprise one or more layer of fabric. In some embodiments, the one or more layers of fabric may allow the opening 510, 511, 512, 513 to remain discreet and relatively unnoticeable. In some implementations, the one or more layers of fabric may form an overlapping layer. In some aspects, the overlapping layer may comprise a sufficient length that the opening 510, 511, 512, 513 remains in a closed state when the opening 510, 511, 512, 513 is not accessed.

In some embodiments, the ostomy-accessible clothing 500, 502, 503 may comprise two or more openings 510, 512, 513. In some implementations, the two or more openings 510, 512, 513 may improve ease of access to the stoma pouch. For example, the stoma pouch may need to switch out for a second stoma pouch, which process may require the use of both hands. The utilization of two or more openings 510, 512, 513 may enable the user to ease manipulate and switch out the stoma bags, utilizing the two or more openings 510, 512, 513 to use both hands for the process.

In some aspects, at least one of the plurality of openings 510, 511, 512, 513 is located on the wearable clothing proximate to the groin, wherein the at least one of the plurality of openings is configured to accept a catheter. This may be particularly useful if the user has a colostomy and may be bed ridden, temporarily or permanently. An opening 510, 511, 512, 513 near medical access points may allow a user to more easily manage their medical devices, particularly as they sleep or when attended to by a medical care provider.

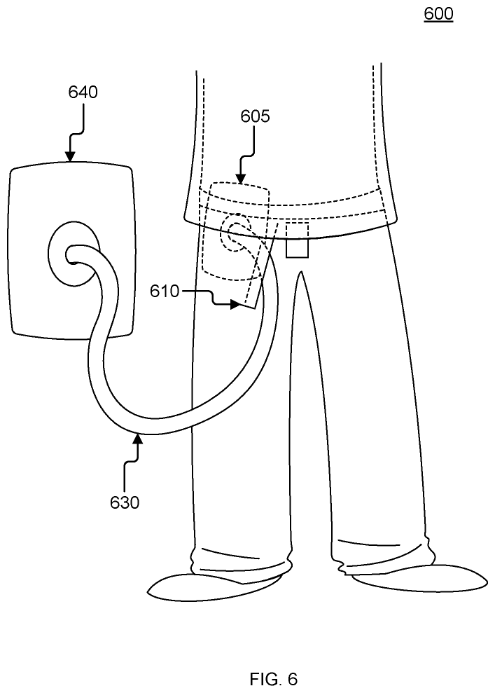

Referring now to FIG. 6, an exemplary ostomy-accessible clothing providing an interface between a stoma pouch and an external device is illustrated. In some embodiments, the ostomy-accessible clothing 600 may comprise at least one opening 610. In some implementations, the opening 610 may allow a connecting apparatus 630 to connect a stoma pouch 605 to an external device 640.

In some implementations, the opening 610 may comprise a predefined slit in the ostomy-accessible clothing 600 that allows the maintenance and replacement of a stoma pouch 605. Because the stoma pouch 605 extends from the lower abdominal to the pelvic region, accessing the stoma pouch 605 in regular clothing is difficult and often requires the adjustment of clothing. Due to the physical location of the stoma pouch 605, adjustment of the clothing to reach the stoma pouch 605 may result in indecency, especially in public settings.

In some aspects, the opening 610 may be positioned adjacent to the location of the stoma pouch 605. In some embodiments, the width of the opening 610 may comprise a predefined width at least the size of a stoma pouch 605. In some implementations, the location and size of the opening 610 may allow the user to access the stoma pouch 605 without clothing adjustment. This may preserve the user's modesty in addition to improving the ease of access to the stoma pouch 605.

In some aspects, the opening 610 may comprise one or more layer of fabric. In some embodiments, the one or more layers of fabric may allow the opening 610 to remain discreet and relatively unnoticeable. In some implementations, the one or more layers of fabric may form an overlapping layer. In some aspects, the overlapping layer may comprise a sufficient length that the opening 610 remains in a closed state when the opening 610 is not accessed.

In some aspects, the opening 610 may allow an external device 640 to interface with the stoma pouch 605. For example, a supplemental pouch may be stored next to a bed to utilize increased storage capacity during sleep. The supplemental pouch may comprise tubing that connects to the stoma pouch 605. To further illustrate the example, ostomy patients typically utilize drainage bags which oftentimes have longer tubes. By utilizing the opening the drainage bag may pass through the opening 610 to connect to the stoma pouch 605.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. An ostomy-accessible clothing, comprising:
a wearable clothing configured to at least partially cover a first stoma of a user;
at least a first opening on the wearable clothing, wherein, when the ostomy-accessible clothing is worn, at least the first opening is located proximate to the first stoma, and wherein at least the first opening further comprises:
at least two layers of fabric, wherein at least the two layers of fabric are configured to overlap when in a resting position and configured to provide at least the first opening when at least the two layers are separated.

2. The ostomy-accessible clothing of claim 1, wherein at least the first opening further comprises a fastening mechanism comprising a first coupling portion on a first of the at least two layers of fabric and a second coupling portion on a second of the at least two layers of fabric, wherein the first coupling portion is connectable to the second coupling portion.

3. The ostomy-accessible clothing of claim 2, wherein the at least one fastening mechanism comprises a hook and loop mechanism.

4. The ostomy-accessible clothing of claim 2, wherein the at least one fastening mechanism comprises a magnetic mechanism.

5. The ostomy-accessible clothing of claim 1, wherein the ostomy-accessible clothing comprises a pocket located on the wearable clothing proximate to at least the first opening.

6. The ostomy-accessible clothing of claim 1, wherein the ostomy-accessible clothing comprises one or more storage compartments.

7. The ostomy-accessible clothing of claim 1, wherein the wearable clothing comprises pants.

8. The ostomy-accessible clothing of claim 7, wherein the ostomy-accessible clothing comprises an elastic portion proximate to at least the first stoma, wherein the elastic portion secures a position of a drainage bag tube or a stoma pouch.

9. The ostomy-accessible clothing of claim 8, wherein the elastic portion maintains alignment of a stoma pouch attached to the first stoma.

10. The ostomy-accessible clothing of claim 1, wherein the wearable clothing comprises a dress.

11. The ostomy-accessible clothing of claim 1, wherein the wearable clothing comprises sleepwear.

12. An ostomy-accessible clothing, comprising:
a wearable clothing configured to at least partially cover at least a first stoma and a medical access point of a user;
a plurality of openings on the wearable clothing, wherein, when the ostomy-accessible clothing is worn, each of the plurality of openings is located proximate to one of the first stoma or the medical access point, and wherein each of the openings further comprises:
at least two layers of fabric, wherein at least the two layers of fabric are configured to overlap when in a resting position and configured to provide each of the plurality of openings when at least the two layers are separated.

13. The ostomy-accessible clothing of claim 12, wherein the wearable clothing covers an arm of the user when worn.

14. The ostomy-accessible clothing of claim 13, wherein the medical access point comprises a location proximate to an insulin patch attached to the user.

15. The ostomy-accessible clothing of claim 12, wherein at least one of the plurality of openings is located on the wearable clothing proximate to a second stoma.

16. The ostomy-accessible clothing of claim 12, wherein the wearable clothing covers a groin of the user when worn.

17. The ostomy-accessible clothing of claim 16, wherein at least one of the plurality of openings is located on the wearable clothing proximate to the groin, wherein the at least one of the plurality of openings is configured to accept a catheter.

18. The ostomy-accessible clothing of claim 12, wherein the wearable clothing comprises sleepwear.

19. The ostomy-accessible clothing of claim 12, wherein the wearable clothing comprises an elastic portion proximate to at least the first stoma, wherein the elastic portion secures a position of a drainage bag tube or a stoma pouch.

20. The ostomy-accessible clothing of claim 19, wherein the elastic portion surrounds at least the first stoma.
